(19) United States
(12) Patent Application Publication
(10) Pub. No.: US 2023/0346729 A1
(43) Pub. Date: Nov. 2, 2023

(54) USE AS AN EMULSION BASE IN A COSMETIC OR PHARMACEUTICAL FORMULATION, OF A COMPOSITION COMPRISING STEAROYL GLUTAMATE

(71) Applicant: GATTEFOSSE SAS, Saint Priest Cedex (FR)

(72) Inventors: Maxime Nollet, Lyon (FR); Hakime Boulghobra, Meyzieu (FR); Jean-David Rodier, Villeurbanne (FR)

(21) Appl. No.: 18/121,748

(22) Filed: Mar. 15, 2023

(30) Foreign Application Priority Data
Mar. 15, 2022 (FR) 2202247

(51) Int. Cl. A61K 31/198; A61K 47/44; A61K 8/44; A61K 8/92

(52) U.S. Cl. CPC A61K 31/198; A61K 47/44; A61K 8/44; A61K 8/92

(57) ABSTRACT

A use, as an emulsifying base in a cosmetic or pharmaceutical formulation, of a composition comprising a mixture of alkali metal stearoyl glutamate and stearoyl glutamic acid, characterised in that the surfactant has a pH of between 7.8 and 9.2, measured at 25° C. in a 5% by mass aqueous solution, and in that the composition comprises between 0.2 g and 0.5 g of aqueous phase/g of surfactant.

Method of manufacturing the emulsifying base and cosmetic or pharmaceutical composition containing it.

### TECHNICAL FIELD

The invention relates to the use, as an emulsifying base in a cosmetic or pharmaceutical formulation, of a composition comprising, as a surfactant, stearoyl glutamate, that is to say, a mixture of alkali metal stearoyl glutamate and stearoyl glutamic acid, a fatty phase and an aqueous phase. The invention also relates to a cosmetic or pharmaceutical formulation incorporating such an emulsifying base, as well as to a method of manufacturing said emulsifying base.

### PRIOR ART

A certain number of emulsifying bases have been developed for the cosmetic or pharmaceutical market. Their objective is to enter into cosmetic or pharmaceutical formulations in the form of an emulsion.

The Applicant's document WO2007/042723 describes an emulsifying base comprising a wax derivative obtained by the reaction of jojoba wax, rice wax, candelilla wax and polyglycerol. The base further comprises cetostearyl alcohol, glycerol stearate and, as a surfactant, sodium stearoyl lactylate.

Even if this base imparts satisfactory emulsifying properties to the compositions which contain it, the stability of the surfactant on storage before preparing the base, and in particular, the risk of hydrolysis of the lactylate group, remains problematic. Furthermore, the presence of a poor-quality lactylate derivative may impart an undesirable odour to the emulsifier.

Document US2008/249192A1 describes an emulsifying base comprising an anhydrous wax containing (a) a compound selected from the esters of pentaerythritol, dipentaerythritol and tripentaerythritol and (b) at least one ester of glycerol and of 012-24 fatty acids, the mass ratio between (a) and (b) being from 1:3 to 3:1. The base further contains, as a surfactant, monosodium or disodium stearoyl glutamate.

The commercially available commercial product sold under the name of sodium stearoyl glutamate (SSG) is an ionic surfactant composed of an acid form (stearoyl glutamic acid) and an ionic basic form (sodium stearoyl glutamate), the ratio of which varies according to the origin or the supplier of the raw material.

The substitution of sodium stearoyl lactylate by stearoyl glutamate makes it possible to solve the odour and hydrolysis problems, but it nevertheless has certain drawbacks.

The first of these is that sodium stearoyl glutamate is insoluble in the fatty phases, whereas stearoyl glutamic acid is insoluble, which requires constant stirring to prevent the particles from sedimenting, and it is essential to obtain a homogeneous mixture.

This problem of homogeneity also makes it impossible to obtain divided solid forms, for example, pellets or flakes, which are themselves of homogeneous composition. In practice, to manufacture pellets, a pelletiser must be used and supplied with a homogeneous liquid free of particles in suspension. Moreover, the presence of these particles may contribute to closing the orifices and make pelletising impossible. Moreover, the passage from the reactor to the pelletiser takes place via non-agitated transfer zones, in which the SSG has a tendency to sediment.

The Applicant has also observed that the emulsifying performance of stearoyl glutamate varied from one raw material to another, making it difficult to reproduce the emulsifying power of the bases incorporating them from one batch to another or from one supplier to another.

More precisely, the Applicant has observed that stearoyl glutamate, depending on its origin, does not have the same surfactant performances, providing the formulations with different levels of viscosity and stability.

Document WO 2019/042999 describes sunscreen spray compositions each comprising a mixture of four or five phases, one of the phases comprising sodium stearoyl glutamate. The pH of the final cosmetic formulations is adjusted by using a tris buffer, once the phases have been mixed.

Document US 2020/306151 describes the ability of an ultra-micro emulsion to be thickened in the presence of a cellulose derivative and of non-ionic surfactants to form a cosmetic composition intended to be applied to the skin. The ultra-micro emulsion contains sodium stearoyl glutamate. The composition contains sodium hydroxide which is used to adjust its pH once the various phases have been mixed.

Document FR3080766A1 describes a cosmetic hair care composition comprising a wax derivative obtained by the reaction of simultaneous transesterification of a mixture comprising jojoba wax and sunflower wax, in the presence of polyglycerol-3. Examples 12 and 13 respectively describe a moisturising night cream for hair and a fortifying hair mask comprising stearoyl glutamate, in addition to the wax derivative. The combination of wax and stearoyl glutamate is not described as an emulsifying base.

Consequently, the invention proposes overcoming the problem of developing an emulsifying base, which is based on stearoyl glutamate and does not have the aforementioned drawbacks.

In other words, the invention proposes overcoming the problem of developing an emulsifying base, which is based on stearoyl glutamate, a fatty phase and an aqueous phase:
whose liquid form as well as the solid form is homogeneous;
which has equivalent emulsifying performances, whatever the origin of the stearoyl glutamate, that is to say, which makes it possible to obtain a clear emulsion from the emulsifying base, without the need to readjust the pH of said emulsion except for cases in which the final emulsion contains ingredients capable of modifying the pH such as, for example, acids.

### DISCLOSURE OF THE INVENTION

The Applicant has observed that the combination of a succession of stirring and resting steps and of a precise proportion of aqueous phase allowing the degree of neutralisation of the stearoyl glutamate to be adjusted, makes it possible to achieve, after mixing all the constituents, a perfect dissolution of the sodium stearoyl glutamate in the fatty phase.

The Applicant has also observed that constant emulsifying performances are obtained, whatever the origin of the stearoyl glutamate, from the moment when the pH of the stearoyl glutamate in a 5% by mass solution in water is between 7.8 and 9.2.

The Applicant thus hypothesises that the acid form (stearoyl glutamic acid)/basic form (sodium stearoyl glutamate) ratio contained in the stearoyl glutamate had an impact on the surfactant performances of the stearoyl glutamate when it was incorporated into an emulsion.

In other words, the invention relates to the use, as emulsifying base in a cosmetic or pharmaceutical formulation, of a composition comprising a fatty phase, an aqueous phase and a surfactant consisting of a mixture of alkali metal stearoyl glutamate and of stearoyl glutamic acid.

The invention is characterised in that the stearoyl glutamate has a pH of between 7.8 and 9.2 measured at 25° C. in a 5% by mass aqueous solution.

In other words, the Applicant has observed that it is necessary to measure and, where appropriate, to adjust the acid form/basic form ratio of the stearoyl glutamate so as to obtain a pH of between 7.8 and 9.2 under the above conditions to provide a stearoyl glutamate whose emulsifying performances are robust and reproducible.

The Applicant has also observed that measuring only the pH of the stearoyl glutamate but not the pH of the emulsifying base had the unexpected advantage of being able to adjust the pH of said base much more precisely. According to a first characteristic, the alkali metal stearoyl glutamate is sodium or potassium.

In a preferred embodiment, the surfactant contains sodium stearoyl glutamate.

According to another characteristic, the surfactant represents between 5% and 12%, advantageously between 7% and 9% by mass of the emulsifying base, surfactant concentrations at which the emulsifying base fulfils the best effects in terms of viscosity and stability, in particular.

These concentrations of surfactant make it possible to obtain a clear emulsion from the emulsifying base, without needing to readjust the pH, except for the cases, as already mentioned, in which certain ingredients disturb the pH of the emulsion, due to their acidic or basic nature.

To ensure satisfactory solubilisation of the stearoyl glutamate, the aqueous phase represents between 1% and 5%, advantageously between 2% and 3.5% by mass of the emulsifying base. Below these values, the Applicant has observed that either there is not enough aqueous phase to allow complete solubilisation of the stearoyl glutamate in the fatty substance, or all of the stearoyl glutamate is certainly solubilised but an excess of aqueous phase appears and settles at the bottom of the mixer.

In practice, the composition comprises between 0.2 g and 0.5 g of aqueous phase/g of surfactant.

According to the invention, the fatty phase contains fatty substances selected from the list comprising such

In a particular embodiment, the fatty phase comprises one or more fatty substances which are solid at ambient temperature, in practice, the melting point of which is greater than 50° C.

Preferably, the fatty substances, whose melting point is greater than 50° C., are selected from the group comprising fatty alcohols, partial glycerides and/or vegetable wax derivatives modified with polyglycerol.

In a preferred embodiment, the fatty phase comprises a wax derivative obtained by the reaction of simultaneous transesterification of a mixture comprising jojoba wax and sunflower wax, in the presence of polyglycerol-3.

In practice, the wax derivative represents between 20% and 40%, preferably between 24% and 35% by mass of the fatty phase.

Advantageously, the fatty phase further comprises at least one C₁₆-C₁₈ fatty alcohol and glycerol palmitostearate.

In practice, the at least one C₁₆-C₁₈ fatty alcohol and the glycerol palmitostearate represent respectively between 35% and 50% by mass of the fatty phase and between 20% and 30% by mass of the fatty phase.

In a particular embodiment, given that the emulsifying base of the invention is homogeneous in hot liquid form (that is to say when the melting point of the fatty phase is greater than 50° C.), the latter may be easily pelletised so as to be in the form of individualised solid particles and in particular in the form of flakes or pellets with a size greater than or equal to 1 millimetre.

The invention also relates to a cosmetic or pharmaceutical formulation, in particular a topical formulation, comprising the emulsifying base as described above.

In practice, the emulsifying base represents between 1% and 8%, preferably between 2% and 5% by mass of the composition.

The emulsifying base of the invention may be implemented in any of the galenical forms normally used for topical application to the skin, lips or hair, in particular in a cosmetic composition in the form of an oil-in-water or water-in-oil or multiple emulsion, a silicone emulsion, or a micro-emulsion or nano-emulsion.

The composition may be fluid to a greater or lesser extent and have the appearance, inter alia, of a white or coloured cream, an ointment, a milk, a lotion or a serum. The composition may also be contained in an aerosol and may be in the form of a foam, spray or stick.

The composition may contain adjuvants which are in normal use in the cosmetic and dermatological fields such as fats, emulsifiers and co-emulsifiers, hydrophilic or lipophilic gelling agents, hydrophilic or lipophilic active ingredients, preservatives, antioxidants, solvents, fragrances, fillers, hydrophilic and lipophilic sunscreens, colourants, bases and acids, propenetrating agents, or polymers.

The quantities of these various adjuvants are those which are in conventional use in the fields under consideration and, for example, 0.01% to 40% of the total mass of the composition. Depending on their nature, these adjuvants may be introduced in the oily phase or in the aqueous phase.

Fats which may be used in the cosmetic composition of the invention are mineral oils, oils of animal origin (lanolin), vegetable oils, synthesised oils (isopropyl myristate, octyldodecanol, isostearyl isostearate, decyl oleate, isopropyl palmitate, caprylic/capric triglycerides), and silicone oils (cyclomethicone, dimethicone). Fats which may be used are fatty alcohols, fatty acids, waxes, butters, hydrogenated oils and gums, in particular, silicone elastomers.

Examples of emulsifiers and co-emulsifiers which may be used in the cosmetic composition of the invention and which may be mentioned are non-ionic, ionic (anionic or cationic) or zwitterionic surfactants, such as, for example, esters of polyglycerols and fatty acids, esters of saccharose and fatty acids, esters of sorbitan and fatty acids, esters of fatty acids and polyoxyethylenated sorbitan, ethers of fatty alcohols and PEG, esters of glycerol and fatty acids, alkyl sulphates, alkyl ether sulphates, alkyl phosphates, alkyl polyglucosides, dimethicone copolyols, and citric acid derivatives.

With regard to hydrophilic gelling agents, particular mention may be made of carboxyvinyl polymers, acrylic copolymers (carbomers), such as acrylate/alkylacrylate copolymers, 2-acrylamido-2-methylpropanoic acid copolymers, polyacrylamides, polysaccharides such as xanthan gum, guar gum, carrageenans, natural gums such as cellulose gum and its derivatives, starches and their derivatives, and clays.

With regard to lipophilic gelling agents, mention can be made of modified clays such as bentonites, metallic salts of fatty acids, silica and its derivatives, ethylcellulose, polyamides and esters of dextrose.

The cosmetic composition may also contain active ingredients. Active ingredients which may in particular be used are depigmenting agents, moisturising agents, anti-oxidants, anti-seborrheic agents, anti-inflammatories, anti-acne agents, keratolytic and/or desquamating agents, anti-wrinkle and tightening active ingredients, draining active ingredients, anti-irritant agents, soothing agents, slimming agents such as xanthan bases (caffeine), vitamins and their mixtures, mattifying agents, anti-aging agents such as retinol, and also essential oils.

Preservatives which may be used according to the invention and which may be mentioned include benzoic acid, its salts and its esters, sorbic acid and its salts, parabens, their salts and esters, triclosan, imidazolidinyl urea, phenoxyethanol, DMDM hydantoin, diazolidinyl urea, chlorphenesin, benzyl alcohol, dehydroacetic acid, levulinic acid and derivatives, salicylic acid and its salts, ethylhexylglycerin, organic acids such as gluconolactone.

Antioxidants which may be used according to the invention that may be mentioned include chelating agents such as EDTA and its salts.

Solvents which may be used according to the invention and which may be mentioned include water, ethanol, glycerin, propylene glycol, butylene glycol, propanediol, pentylene glycol and hexanediol.

Fillers which may be used according to the invention and which may be mentioned include talc, kaolin, mica, serecite, magnesium carbonate, aluminium silicate, magnesium silicate, organic powders such as nylon, PMMA, starches and natural powders such as rice powder. Such fillers may also be chemically modified or coated to modify their characteristics.

Sunscreens which may be used according to the invention and which may be mentioned include UVA and UVB sunscreens which are in conventional use, such as benzophenones, butyl methoxydibenzoyl methane, octocrylene, octyl methoxycinnamate, 4-methylbenzylidene camphor, octyl salicylate, tacephthalylidene dicamphor sulphonic acid, phenylbenzimidazolesulphonic acid, homosalate, 2-(4-diethylamino-2-hydroxybenzoyl)benzoate, disodium phenyl dibenzimidazole tetrasulphonate, methylene bis-benzotriazolyl tetramethylbutylphenol, triazine derivatives, polysilicone-15 and drometrizole trisiloxane. The physical sunscreens TiO2 and ZnO in their micrometric and nanometric forms, coated or uncoated, may also be mentioned.

Colourants which may be used according to the invention that may be mentioned include lipophilic colourants, hydrophilic colourants, pigments and nacres in normal use in cosmetic or dermatological compositions, and their mixtures.

Bases and acids which may be used according to the invention that may be mentioned include sodium hydroxide, triethanolamine, aminomethyl propanol, potassium hydroxide, citric acid, lactic acid and phosphoric acid.

Propenetrating agents which may be used according to the invention that may be mentioned include alcohols and glycols (ethanol, propylene glycol), ethoxydiglycol, alcohols and fatty acids (oleic acid), fatty acid esters, dimethyl isosorbide.

The composition according to the invention may be used as a care product, as a cleansing product and/or as a product for making up the skin, as a sunscreen product, or as a hair care product.

The invention also relates to a method of manufacturing the emulsifying base, described above, in which:
the fatty phase is prepared in the liquid state;
the surfactant is introduced in the fatty phase, in practice, all of the surfactant, through stirring;
before or after adding the surfactant and before adding water to the fatty phase, the pH at 5% in water, at 25° C., of the stearoyl glutamate is measured separately, that is to say independently of the fatty phase;
where appropriate, adjustments are made to the stearoyl glutamic acid/sodium stearoyl glutamate ratio by adjusting the pH of the surfactant to 5%, at 25° C., in water by adding a base or acid to obtain a pH of between 7.8 and 9.2.
when it is necessary to add a base or of an acid, the mass of the base or acid to be added is determined as a function of the quantity of surfactant used when manufacturing the emulsifying base, that is to say the quantity of surfactant introduced in the fatty phase;
the aqueous phase is prepared by adding the mass of base or acid determined in the preceding step;
0.2 g et 0.5 g of aqueous phase/g surfactant are added to the fatty phase;
stirring and resting phases are alternated until the surfactant has completely dissolved.

As already emphasised, the adjusting the pH of the surfactant is more precise by determining the mass of the base or of acid necessary, to then add said mass of base or of acid in the fatty phase rather than adjusting the pH of the emulsifying base once manufactured.

In the embodiment according to which the melting point of the components of the fatty phase is greater than, in practice, 50° C., the step of preparing the fatty phase in the liquid state is performed by heating said components to a temperature greater than their melting point.

According to a preferred embodiment, the stirring phases last between 5 minutes and 15 minutes, preferably 10 minutes, whereas the resting phases last between 2 minutes and 10 minutes, preferably 5 minutes.

Advantageously, the stirring and resting phases are repeated at least twice, preferably 4 times.

In a particular embodiment, in which the melting point of the components of the fatty phase is higher, in practice at 50° C., the emulsifying base is subjected to a pelletising step.

The invention and the resulting advantages will become apparent from the following embodiments, made in support of the accompanying figures:

### BRIEF DESCRIPTION OF THE DRAWINGS

FIGS. 1A-1G is a series of photographs of the products obtained at the end of the various steps of the method of manufacturing the emulsifying base.

FIG. 2 is a photograph of the emulsifying base of the invention in the form of pellets.

### DETAILED DESCRIPTION OF THE INVENTION

Composition of the Emulsifying Base

The composition of the emulsifying base is reproduced in Table 1 below.

TABLE 1

| | | |
|---|---|---|
| Fatty phase | Cetearyl alcohol (C16-18 alcohol) | 45% |
| | Glycerol palmitostearate | 22.5% |
| | Jojoba Esters/Heliantus Annuus (Sunflower) | 22.5% |
| | Seed Wax/polyglycerin-3 (Wax derivatives) | |
| Surfactant | Stearoyl glutamate | 7.0% |
| Aqueous phase | Water that may contain a base or an acid | 3.0% |

Method of Manufacturing the Emulsifying Base

The emulsifying base is prepared as follows.

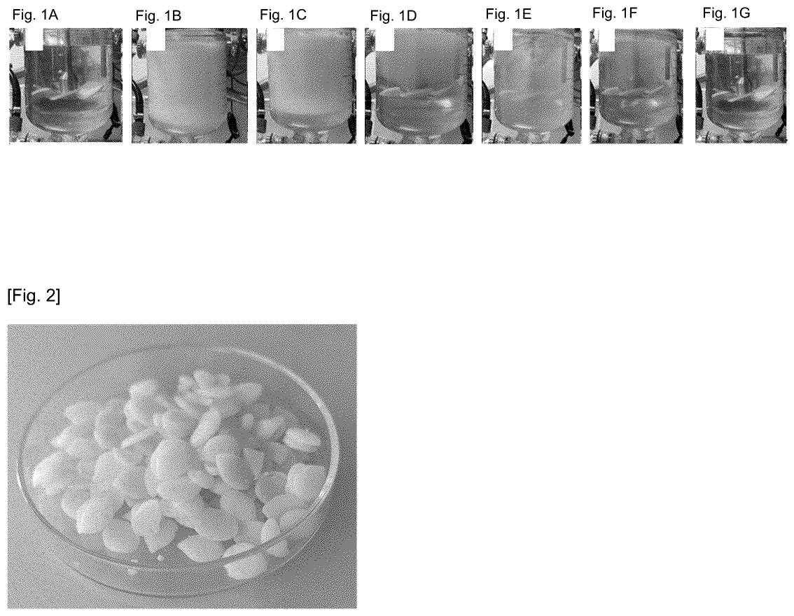

The various compounds of the fatty phase are mixed and the mixture is then melted at 80° C. The liquid phase obtained is then stirred at a rate of 250 rpm for a one litre reactor. FIG. 1A is a photograph of the phase obtained at this stage of the method.

The stearoyl glutamate is then slowly introduced in the fatty phase, while stirring constantly. Stirring the mixture allows good dispersion of the powder. The dispersion is represented in FIG. 1B.

The pH of the stearoyl glutamate is measured separately at 5%, at 25° C., in water. Where appropriate, the stearoyl glutamic acid/sodium stearoyl glutamate ratio is adjusted by adjusting the pH of the surfactant to 5%, at 25° C., in water, by adding a base or acid to obtain a pH of between 7.8 and 9.2. In this case, the base or acid mass to be added is extrapolated to the quantity of surfactant used when manufacturing the emulsifying base. The aqueous phase is prepared by adding the base or acid mass determined above.

The aqueous phase is then added to the dispersion in FIG. 1B. The quantity of aqueous phase added is between 0.2 g and 0.5 g of water/g of stearoyl glutamate. Below 0.2 g of water/g of stearoyl glutamate, there is not enough water to allow complete solubilisation of the stearoyl glutamate in the fatty phase. Above 0.5 g of water/g of stearoyl glutamate, all of the stearoyl glutamate is solubilised, but an excess of water appears and settles at the bottom of the mixer.

The solubilisation of the stearoyl glutamate in the fatty phase is performed in several stirring steps and the stirring is stopped (rest).

Once the water is added, the dispersion, in this example, is stirred for 10 minutes and then left to rest for 5 minutes (FIG. 1C).

This step of stirring for 10 minutes and resting for 5 minutes is repeated for between 1 and 4 times (FIGS. 1D-1F) until a homogeneous clear product is obtained (FIG. 1G).

This homogeneous and clear mixture may be kept at 90° C. for 48 hours in a closed container without any change in macroscopic appearance or chemical composition.

Pelletisation

Once the emulsifying base is clear, it may be pelletised without difficulty to obtain pellets that are homogeneous with each other throughout the entire duration of the pelletising operation, but also within the same pellet (FIG. 2).

Comparative Tests

Three formulations are prepared which differ from one another solely by the pH of the surfactant measured at 5% in water. The emulsifying base is that of Table 1, after adjusting or not adjusting the pH of the surfactant to 5% in water.

The composition of the formulations is shown in Table 2. The proportions of the ingredients are given as a percentage by mass.

TABLE 2

| | | | Formu- | Formu- | Formu- |
|---|---|---|---|---|---|
| | | | lation | lation | lation |
| | | Ingredient | 1 | 2 | 3 |
| Fatty | | Hydrogenated | 4.5% | 4.5% | 4.5% |
| Phase | | polydecene | | | |
| | | Triglicerides in C8/C10 | 4.5% | 4.5% | 4.5% |
| | | Isostearyl isostearate | 3% | 3% | 3% |
| | | Apricot kernel oil | 3% | 3% | 3% |
| | | Phenoxyethanol | 0.72% | 0.72% | 0.72% |
| | | Ethylhexylglycerin | 0.08% | 0.08% | 0.08% |
| Emulsifier | | Emulsifier according to | 6% | | |
| | | Table 1 in compliance | | | |
| | | with the invention (pH at | | | |
| | | 5% in water containing | | | |
| | | the surfactant between | | | |
| | | 7.8 and 9.2) | | | |
| | | Emulsifier according to | | 6% | |
| | | Table 1 in compliance | | | |
| | | with the invention (pH at | | | |
| | | 5% in water containing | | | |
| | | the surfactant <7.8) | | | |
| | | Emulsifier according to | | | 6% |
| | | Table 1 in compliance | | | |
| | | with the invention (pH at | | | |
| | | 5% in water containing | | | |
| | | the surfactant >9.2) | | | |
| Aqueous | | Water | 78.2% | 78.2% | 78.2% |
| phase | | | | | |
| 24 | h | Appearance | White | White | White |
| | | | cream, | cream, | cream, |
| | | | glossy | glossy | glossy |
| | | Viscosity (cP) | 52000 | 40800 | 25600 |
| | | pH | 7.3 | 6.3 | 7.8 |
| 1 | week | Appearance | Con- | Con- | Con- |
| | | | forming | forming | forming |
| | | Viscosity (cP) | 56300 | 38600 | 30600 |
| | | pH | 7.4 | 6.3 | 7.8 |
| | | Appearance at 40° | Con- | Con- | Con- |
| | | | forming | forming | forming |
| 1 | month | Appearance | Con- | Con- | Con- |
| | | | forming | forming | forming |
| | | Viscosity (cP) | 51500 | 39600 | 22800 |
| | | pH | 7.6 | 6.3 | 7.1 |
| | | Appearance at 40° | Con- | Non- | Con- |
| | | | forming | con- | forming |
| | | | | forming | |
| | | | | Start of | |
| | | | | creaming | |
| | | | | (2 s) | |
| 3 | Months | Appearance | Con- | Con- | Con- |

| | | | |
|---|---|---|---|
| | ing | forming | forming |
| Viscosity (cP) | 47300 | 31400 | 14400 |
| | | | Non |
| | | | con- |
| | | | forming |
| pH | 7.5 | 6.3 | 6.8 |
| | | | Non |
| | | | con- |
| | | | forming |
| Appearance at 40° | Con- | Non | Con- |
| | forming | con- | forming |
| | | forming | |

It is clear from the above that formula 1, whose pH at 5% in water of the surfactant is between 7.8 and 9.2, has a viscosity close to 50,000 cP, which corresponds to what is expected. The results also show that the formula 1 has a good level of stability over time.

Examples of Cosmetic Formulations

Anti-Aging Serum

TABLE 3

| Ingredient | % m/m |
|---|---|
| Deionised water | QSP 100.0 |
| Hydroxypropyl guar | 1.0 |
| Glycerin | 2.0 |
| Xanthan gum | 0.2 |
| Pullulan | 2.0 |
| EMULSIFYING BASE ACCORDING TO THE | 3.0 |
| INVENTION | |
| Preservative | 0.7 |
| Undecane, tridecane | 10.0 |
| Anti-aging agent | 1.0 |
| Nacres | 0.5 |
| Citric acid | |

Sun Body Lotion SPF 30

TABLE 4

| Ingredient | % m/m |
|---|---|
| Deionised water | Qsp 100.00 |
| Sphingomonas Ferment Extract | 0.15 |
| Ethylhexyl Triazone | 3.00 |
| Diethylamino Hydroxybenzoyl Hexyl Benzoate | 5.00 |
| Bis-Ethylhexyloxyphenol Methoxyphenyl Triazine | 2.00 |
| Ethylhexyl Salicilate | 5.00 |
| Diisopropyl adipate, Ethylcellulose | 1.50 |
| Dibutyl Adipate | 6.00 |
| Diisopropyl Sebacate | 9.00 |
| Butyl Octyl Salicilate | 2.50 |
| Phenoxyethanol, Ethylhexylglycerin | 0.70 |
| Glycerol behenate | 1.50 |
| EMULSIFYING BASE ACCORDING TO THE | 5.00 |
| INVENTION | |
| Diisostearoyl polyglyceryl-3 dimer dilinoleate | 2.00 |
| Copemica cerifera (carnauba), wax, oryza sativa | 3.00 |
| (rice) bran wax | |
| Butylene glycol | 4.00 |
| Moisturising agent | 2.00 |
| Citric acid | qs |

Day cream

TABLE 5

| Ingredient | % m/m |
|---|---|
| Cetyl alcohol | 3.0 |
| Jojoba Esters/Heliantus Annuus (Sunflower) Seed | 2.0 |
| Wax, acacia decurrens fower wax, polyglycerin-3 | |
| C10-18 triglycerides | 5.0 |
| Dicaprylyl ether | 18.0 |
| Vegetable hydrogenated oil | 5.0 |
| Preservative | 1.0 |
| EMULSIFYING BASE ACCORDING TO THE | 5.0 |
| INVENTION | |
| Deionised water | Qsp 100.0 |
| Acacia Senegal gum, xanthan gum | 0.4 |
| Glycerin | 5.0 |
| Citric acid | 0.5 |
| Tightening agent | 1.0 |
| Fragrance | 0.2 |
| Nacres | 1.2 |
| Mattifying agent | 2.0 |

1. An emulsifying base in a cosmetic or pharmaceutical formulation, said emulsifying base comprising:
a fatty phase, an aqueous phase, and a surfactant containing a mixture of alkali metal stearoyl glutamate and stearoyl glutamic acid, wherein the surfactant has a pH of between 7.8 and 9.2, measured at 25° C. in a 5% by mass solution in water, and in that the composition comprises between 0.2 g and 0.5 g of aqueous phase/g of surfactant.

2. The emulsifying base according to claim 1, wherein the alkali metal is sodium.

3. The emulsifying base according to claim 1, characterised in that the alkali metal stearoyl glutamate is sodium stearoyl glutamate.

4. The emulsifying base according to claim 1, wherein the surfactant represents between 5% and 12%, advantageously between 7% and 9% by mass of the emulsifying base.

5. The emulsifying base according to claim 1, wherein the fatty phase comprises a wax derivative obtained by the reaction of simultaneous transesterification of a mixture comprising jojoba wax and sunflower wax in the presence of polyglycerol-3.

6. The emulsifying base according to claim 5, wherein the wax derivative represents between 20% and 40% by mass of the fatty phase.

7. The emulsifying base according to claim 5, wherein the fatty phase further comprises at least one C16-C18 fatty alcohol and glycerol palmitostearate.

8. The emulsifying base according to claim 7, wherein the at least one C16-C18 fatty alcohol and the glycerol palmitostearate represent respectively between 35% and 50% by mass of the fatty phase and 20% to 30% by mass of the fatty phase.

9. The emulsifying base according to claim 1, wherein the composition is in the form of individualised solid particles.

10. The emulsifying base according to claim 9, wherein the individualised particles are in the form of flakes or pellets with a which are larger than or equal to 1 mm.

11. The emulsifying base according to claim 1, wherein the emulsifying base is included in a cosmetic composition.

12. The cosmetic composition according to claim 11, wherein the emulsifying base represents between 1% and 8% by mass of the composition.

13. A method of manufacturing an emulsifying base for a cosmetic or pharmaceutical formulation comprising a fatty phase, an aqueous phase, and, as surfactant, a mixture of sodium stearoyl glutamate and stearoyl glutamic acid, comprising:
preparing the fatty phase in the liquid state;
introducing the surfactant in the fatty phase, through stirring;
measuring the pH of the stearoyl glutamate at 5% in water separately at 25° C., before or after adding the surfactant, and before adding the water to the fatty phase;
where appropriate, adjusting the stearoyl glutamic acid/sodium stearoyl glutamate ratio by adjusting the pH of the surfactant to 5% in water, at 25° C., by adding a base or acid to obtain a pH of between 7.8 and 9.2;
when it is necessary to add a base or an acid, determining the mass of the base or acid to be added as a function of the quantity of surfactant used when manufacturing the emulsifying base;
preparing the aqueous phase by adding the mass of base or acid determined in the preceding step,
adding between 0.2 g and 0.5 g of aqueous phase/g of surfactant to the fatty phase;
alternating the stirring and resting phases until the surfactant has completely dissolved.

14. The method according to claim 13, wherein the emulsifying base is subjected to a pelletising step.

15. The emulsifying base according to claim 5, wherein the wax derivative represents between 24% and 35% by mass of the fatty phase.

16. The cosmetic composition according to claim 11, wherein the emulsifying base represents between 2% and 5% by mass of the composition.
